SEQUENCE LISTING (22 sequences)

SEQ ID NO: 1 - protein, 427 aa, synthetic construct
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 2 - protein, 427 aa, synthetic construct
TQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 3 - protein, 427 aa, synthetic construct
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYSVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 4 - protein, 427 aa, synthetic construct
TQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVNEKYLSEFCFSGTYILSLLLQGYHFTADSWEHIHFIGKIQGSDAGWTLGYMLNLTNMIPAEQPLSTPLSHST

SEQ ID NO: 5 - protein, 427 aa, synthetic construct
TQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLLQGYHFTADSWEHIHFIGKIQGSDAGWTLGYMLNLTNMIPAEQPLSTPLSHST

SEQ ID NO: 6 - protein, 427 aa, synthetic construct
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLQQGYHFTADSWEHIHFIGKIQGSDAGWTLGYMLNLTNMIPAEQPLSTPLSHST

SEQ ID NO: 7 - protein, 427 aa, synthetic construct
TQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLQQGYHFTADSWEHIHFIGKIQGSDAGWTLGYMLNLTNMIPAEQPLSTPLSHST

SEQ ID NO: 8 - protein, 427 aa, synthetic construct
TQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYSVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 9 - protein, 433 aa, synthetic construct
APTSSSTQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLLQGYHFTADSWEHIHFIGKIQGSDAGWTLGYMLNLTNMIPAEQPLSTPLSHST

SEQ ID NO: 10 - protein, 433 aa, synthetic construct
APTSSSTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLQQGYHFTADSWEHIHFIGKIQGSDAGWTLGYMLNLTNMIPAEQPLSTPLSHST

SEQ ID NO: 11 - protein, 433 aa, synthetic construct
APTSSSTQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLQQGYHFTADSWEHIHFIGKIQGSDAGWTLGYMLNLTNMIPAEQPLSTPLSHST

SEQ ID NO: 12 - protein, 430 aa, synthetic construct
APTTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLQQGYHFTADSWEHIHFIGKIQGSDAGWTLGYMLNLTNMIPAEQPLSTPLSHST

SEQ ID NO: 13 - protein, 430 aa, synthetic construct
APTTQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLQQGYHFTADSWEHIHFIGKIQGSDAGWTLGYMLNLTNMIPAEQPLSTPLSHST

SEQ ID NO: 14 - protein, 430 aa, synthetic construct
APTTQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 15 - protein, 430 aa, synthetic construct
APTTQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYSVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 16 - protein, 443 aa, synthetic construct
APTSSSTKKTQLTSSGTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 17 - protein, 442 aa, synthetic construct
APTSSSTKKTQLTSSTQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVNEKYLSEFCFSGTYILSLLLQGYHFTADSWEHIHFIGKIQGSDAGWTLGYMLNLTNMIPAEQPLSTPLSHST

SEQ ID NO: 18 - protein, 433 aa, synthetic construct
APTSSSTQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYSVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 19 - protein, 430 aa, synthetic construct
APTTQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLLQGYHFTADSWEHIHFIGKIQGSDAGWTLGYMLNLTNMIPAEQPLSTPLSHST

SEQ ID NO: 20 - protein, 430 aa, synthetic construct
APTTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 21 - protein, 430 aa, synthetic construct
APTTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYSVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 22 - protein, 510 aa, Homo sapiens
MEDTKESNVKTFCSKNILAILGFSSIIAVIALLAVGLTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYVFLMVLFSLVLFTVAIIGLLIFHKPSYFWKDMV

(19) United States
(12) Patent Application Publication
(10) Pub. No.: US 2026/0345746 A1
(43) Pub. Date: Nov. 5, 2026

(54) TREATMENT FOR ACUTE ORGAN INJURY USING CD39, RECOMBINANT CD39

(71) Applicant: NOVARTIS AG, Basel (CH)

(72) Inventors: Claudio CALONDER, Basel (CH); Ghionul IBRAM, Randolph, NJ (US); Guido JUNGE, Basel (CH); Matthew James ROWLAND, Oxford (GB); Max Philipp TAUBERT, Baden-Württemberg (DE); Max WARNCKE, Baden-Württemberg (DE); Hermann Markus WEISS, Baden-Württemberg (DE); Yinong ZHOU, Bridgewater, NJ (US)

(21) Appl. No.: 18/998,941

(22) PCT Filed: Jul. 27, 2023
(86) PCT No.: PCT/IB2023/057612

§ 371 Date: Jan. 27, 2025

Related U.S. Application Data
(60) Provisional application No. 63/369,839, filed on Jul. 29, 2022.

(51) Int. Cl. A61K 38/46; A61K 45/06; A61P 13/12

(52) U.S. Cl. CPC A61K 38/46; A61K 45/06; A61P 13/12; C12Y 306/01005

(57) ABSTRACT

The present invention pertains to the use for treating organ injury, in particular acute organ injury, such as acute kidney injury (AKI) using CD39, human recombinant CD39 and variants thereof.

### TECHNICAL FIELD

The present disclosure generally relates to methods for treating organ injury, in particular acute organ injury, such as acute kidney injury (AKI) using CD39, human recombinant CD39 and variants thereof, such as the recombinant CD39 variant which has an amino acid sequence selected from the group consisting of SEQ ID NOs: 1 to 21.

### BACKGROUND OF THE DISCLOSURE

Acute kidney injury (AKI) is a silent condition, characterized by a sudden deterioration of kidney function, sometimes with reduced or even complete loss of urine output. Most patients will experience this syndrome during a hospital stay, especially critically ill patients and patients undergoing major surgeries. Recovery from AKI is possible, however, in some patients kidney function will continue to decline further and remain chronically impaired. Some patients will need at least short-term dialysis, or will be left with severe renal impairment and remain dialysis-dependent with the need for a kidney transplant.

Furthermore, during sepsis a dysregulated host response to infection leads to life-threatening multi organ dysfunction. Sepsis is a leading cause of death and a major public health problem that affects millions of patients worldwide every year. Approximately 90% of sepsis is caused by bacterial infection and 40-60% of septic patients experience acute kidney injury (AKI). Acute kidney injury is a common complication of sepsis. 28% of sepsis patients acquire AKI. Development of acute kidney injury (AKI) in patients with sepsis is associated with significantly increased mortality rate of up to 65%, and survivors are at risk of developing chronic kidney disease (CKD) and end stage renal disease, resulting in a significant health and financial burden for both patient and society. Sepsis associated acute kidney injury (SA-AKI) is a multifactorial syndrome with inflammatory, nephrotoxic, and ischemic insults occurring simultaneously with other pathophysiological responses rapidly leading to kidney impairment. Over the last 30 years, there were a considerable number of potential drug targets and the development of new therapies to treat sepsis and AKI. Attempts were primarily focusing on improving hemodynamics, reducing oxidative stress, and blocking hyper-inflammatory response, however, all of which have failed in clinical trials. Management of patients with SA-AKI relies mainly on antibiotics and supportive treatment including fluid resuscitation, vasoactive agents and renal replacement therapy.

AKI is also a frequent and serious complication of cardiopulmonary bypass surgery (CPBS), cardiac surgery associated AKI (CSA-AKI). AKI is new or worsened renal insufficiency characterized by a relatively abrupt decrease in glomerular filtration rate (GFR), often accompanied by a reduction in urine output. AKI usually occurs within the first 7 days after an initial insult, most commonly following an episode of transient hypotension of any cause, but may also occur in response to nephrotoxins or radio-contrast media. The clinical incidence of AKI is found in 3-18% of all hospitalized adult patients worldwide, and is more common in the context of complex surgery. Depending on the definition, AKI occurs in up to 15-40% of adults after CPBS. Severe AKI requires renal replacement therapy in 1-5% of cases and is associated with a mortality rate of up to 70%.

Several lines of evidence have shown the potential benefits by conversion of extracellular ATP to adenosine in treating sepsis and the related organ injury. In a septic animal model induced by cecal ligation and puncture (CLP), treatment with apyrase, an ATPase that promotes the enzymatic breakdown of extracellular ATP, diminished the concentration of extracellular ATP in peritoneal lavages and significantly reduced mortality.

A clinical study conducted with alkaline phosphatase (ALP) in SA-AKI patients showed that AP improved kidney function determined by a composite endpoint of creatinine clearance, requirement for RRT, and duration of RRT. Alkaline phosphatase is an endogenous enzyme that exerts detoxifying effects through dephosphorylation of endotoxins and extracellular ATP. In a relatively larger clinical study in patients with SA-AKI (N=301), results have suggested a potential mortality benefit with treatment of AP compared to placebo even though the study did not meet its primary endpoint in improving short-term kidney function measured by area-under-the time corrected endogenous creatinine clearance from Day 1 to Day 7.

There is currently no approved pharmacological therapy to treat or prevent AKI, SA-AKI.

### SUMMARY OF THE DISCLOSURE

We have now devised novel treatments for subjects with acute organ injuries in general, and AKI in particular AKI, e.g. sepsis-associated acute kidney Injury (SA-AKI) or cardiac surgery associated AKI (CSA-AKI), using a recombinant CD39 and variants thereof, as defined herein, that are safe, effective and provide sustained responses for patients. These novel treatments satisfy a long-felt need of clinicians and patients for a safe, and effective therapy for AKI. Treatment approaches to enhance extracellular ATP conversion to adenosine using the recombinant CD39 mechanisms of action to hydrolyzing extracellular ATP and ADP to AMP represents a therapeutic strategy to mitigate and dampen acute organ injury in general. Due to the efficient hydrolysis of ATP and ADP, the recombinant CD39, as defined herein, is dose dependently inhibiting ATP/LPS-induced IL-1B secretion as well as ADP and thrombin-induced platelet. Inhibition of thrombin-induced platelet aggregation is attributable to release of ADP from the platelet-dense granules through activation of protease-activated receptor-4 (PAR4) by thrombin inducers. Furthermore, the recombinant CD39, as defined herein inhibits the ATP/LPS-induced IL-1β release in a concentration-dependent manner. Such treatments show anti-inflammation, anti-coagulation and tissue protection that could lead to improvement of renal function and reduction of mortality in subjects suffering from AKI, such as SA-AKI or CSA-AKI.

In one embodiment, a recombinant CD39 is provided, said recombinant CD39 comprising the amino acid sequence of SEQ ID NOs: 1 to 21, and wherein said recombinant CD39 is to be administered to a subject in need thereof, at a dose of from about 0.1 mg/kg to about 10 mg/kg of body weight (mg per kilogram subject body weight (mg/kg)).

In a preferred embodiment, the recombinant CD39 designated CD39* is provided. Specifically, CD39* comprises the amino acid sequence of SEQ ID NO: 21, and wherein said recombinant CD39* is to be administered to a subject in need thereof, at a dose of from about 1 mg/kg to about 5 mg/kg.

In one embodiment, the route of administration is intravenous (IV), IV infusion, of the recombinant CD39 according to the embodiments herein described.

In another example, an appropriate regimen for a recombinant CD39, as defined herein, is a daily regimen.

In some embodiments, the recombinant CD39, as defined herein, preferably CD39*, may be administered to the subject at a dose of from about 0.1 mg/kg to about 10 mg/kg, for example at a dose of about 1 mg/kg, 2 mg/kg, 3 mg/kg, 4 mg/kg or of about 5 mg/kg, delivered IV.

In yet another specific embodiment, a dose of about 4 mg/kg of the recombinant CD39, as defined herein, preferably CD39*, is administered IV, during 2 hours.

In yet another specific embodiment, a dose of about 5 mg/kg of the recombinant CD39, as defined herein, preferably CD39*, is administered IV, during 2 hours.

The recombinant CD39, as defined herein, preferably CD39*, may be administered to the subject daily, e.g. i.v., at a dosing of about 4-5 mg/kg, by infusion, within a number of hours after AKI or sepsis is first diagnosed for said subject.

The recombinant CD39, as defined herein, preferably CD39*, may be administered to the subject, e.g. IV, at a dosing of about 4-5 mg/kg, by infusion within a time interval initiating from suspected or confirmed AKI diagnosis for said subject.

The recombinant CD39, as defined herein, preferably CD39*, may be administered to the subject, e.g. IV, at a dosing of about 4-5 mg/kg, by infusion, within about 48 hours, or less, after AKI is first diagnosed for said subject (e.g., within 12 hours, within 24 hours, or within 36 hours, for example).

In some embodiments, the treatment regimen may stretch over a number of hours to a day to several weeks as determined by a competent caregiver (treating physician).

In one embodiment, the present invention comprises administering the recombinant CD39, as defined herein, preferably CD39*, to a subject with AKI, e.g. SA-AKI or CSA-AKI, in the range of about 0.1 mg/kg to about 10 mg/kg per treatment, preferably in the range of 1 mg/kg to 5 mg/kg, preferably about 4-5 mg/kg per treatment. In one embodiment a subject receives 4 or 5 mg/kg, per treatment. In one embodiment the subject with AKI, e.g. SA-AKI or CSA-AKI, receives the treatment once, within about 48 hours, after AKI e.g. SA-AKI or CSA-AKI, is first diagnosed.

In some embodiments, the recombinant CD39, as defined herein, preferably CD39*, is to be administered in combination with one or more additional agents. In some embodiments, the one or more additional agents is a steroid, a metalloproteinase inhibitor, a serine protease inhibitor, a topical anesthetic emulsion, a spreading factor inhibitor, an anti-nausea agent, or an antibiotic.

In another aspect, the disclosure provides new dosing regimens for the recombinant CD39, as defined herein, preferably CD39*, that can be used in methods of treating or preventing AKI, e.g. SA-AKI or CSA-AKI.

In another aspect, the disclosure provides new dosing regimens for the recombinant CD39, as defined herein, preferably CD39*, that can be used in methods of treating or preventing sepsis.

In another aspect, the disclosure provides new dosing regimens for the recombinant CD39, as defined herein, preferably CD39* for use as a medicament, wherein the recombinant CD39 is to be administered at a dose of from about 0.1 mg/kg to about 10 mg/kg, for example at a dose of about 1 mg/kg, about 2 mg/kg, about 3 mg/kg, about 4 mg/kg or about 5 mg/kg, in a subject in need thereof. In yet another aspect, the disclosure provides new methods of treatment using the recombinant CD39, as defined herein, preferably CD39*, wherein the recombinant CD39 is to be administered to a subject in need thereof, at a dose of from about 0.1 mg/kg to about 10 mg/kg, for example at a dose of about 1 mg/kg, about 2 mg/kg, about 3 mg/kg, about 4 mg/kg or about 5 mg/kg.

### DETAILED DESCRIPTION OF THE DISCLOSURE

For purposes of interpreting this specification, the following definitions will apply and whenever appropriate, terms used in the singular will also include the plural and vice versa.

### EMBODIMENTS

A1. A recombinant CD39 for use in treating or preventing AKI in a subject in need thereof.

A2. The recombinant CD39 according to embodiment A1, wherein the AKI is severe AKI.

A3. The recombinant CD39 according to embodiment A1, wherein at least one symptom of AKI in a subject is reduced or abolished.

A4. The recombinant CD39 according to any one of the above embodiments, wherein the severity of AKI is reduced by at least one stage, as defined by modified AKI Network (AKIN) criteria for serum creatinine.

A5. The recombinant CD39 according to any one of embodiments A1-A4 for use in preventing AKI in a subject.

A6. The recombinant CD39 according to any one of embodiments A1-A4, for use in reducing the risk of AKI.

A7. The recombinant CD39 according to any one of embodiments A1-A4, for use in reducing the severity of AKI.

A8. The recombinant CD39 according to any one of the above embodiments, wherein the incidence of acute kidney disease severity is reduced, evaluated by renal function and Major Adverse Kidney Events (MAKE).

A9. The recombinant CD39 according to any one of the above embodiments, wherein the severity of SA-AKI is reduced.

A10. The recombinant CD39 according to any one of the above embodiments, wherein the progression of AKI, e.g. sepsis-AKI, is prevented.

A11. The recombinant CD39 according to any one of the above embodiments, for use in improving major adverse kidney events, e.g. decreasing renal impairment.

A12. A recombinant CD39 for use in treating or preventing sepsis.

A13. The recombinant CD39 according to embodiment A12, wherein diagnosis of sepsis is performed according to criteria defined by The Third International Consensus.

A14. The recombinant CD39 according to embodiment A12 or A13, wherein sepsis and septic Shock (Sepsis-3) are defined based on:
Suspected or confirmed infection, AND
Acute increase of sequential organ failure score (SOFA) score of 2 or more (excluding renal component). The baseline SOFA score should be assumed to be zero unless the participant is known to have pre-existing (acute or chronic) organ dysfunction before the onset of infection.

A15. The recombinant CD39 according to any one of the above embodiments, is to be administered at a dose of from about 0.1 mg/kg to about 10 mg/kg, for example at a dose of about 1 mg/kg, about 2 mg/kg, about 3 mg/kg, about 4 mg/kg or about 5 mg/kg.

A16. The recombinant CD39 according to any one of the above embodiments, is to be administered at a dose of about 4 mg/kg of the recombinant CD39.

A17. The recombinant CD39 according to any one of the above embodiments, is to be administered at a dose of about 5 mg/kg of the recombinant CD39.

A18. The recombinant CD39 according to any one of the above embodiments, is to be administered IV, e.g. is to be administered IV during about 2 hours.

A19. The recombinant CD39 according to any one of the above embodiments, wherein the recombinant CD39 is to be administered within a number of hours after AKI is first diagnosed for said subject.

A20. The recombinant CD39 according to any one of the above embodiments, wherein the recombinant CD39 is to be administered to the subject daily, e.g. IV, at a dosing of about 4 or 5 mg/kg, by infusion, within a time interval initiating from suspected or confirmed AKI diagnosis for said subject.

A21. The recombinant CD39 according to any one of the above embodiments, wherein the recombinant CD39 is to be administered within about 48 hours, or less, after AKI is first diagnosed for said subject (e.g., within 12 hours, within 24 hours, or within 36 hours, for example).

A22. The recombinant CD39 according to any one of the above embodiments, wherein the recombinant CD39 is to be administered within about 24 hours, or as soon as possible after AKI, or sepsis, is first diagnosed.

A23. The recombinant CD39 according to any one of the above embodiments, wherein the recombinant CD39 is to be administered within a number of hours after sepsis is first diagnosed for said subject.

A24. The recombinant CD39 according to any one of the above embodiments, wherein the recombinant CD39 is to be administered to the subject daily, e.g. IV, at a dosing of about 4 or 5 mg/kg, by infusion, within a time interval initiating from suspected or confirmed sepsis diagnosis for said subject.

A25. The recombinant CD39 according to any one of the above embodiments, wherein the recombinant CD39 is to be administered within about 48 hours, or less, after sepsis is first diagnosed for said subject (e.g., within 12 hours, within 24 hours, or within 36 hours, for example).

A26. The recombinant CD39 according to any one of the above embodiments, wherein the recombinant CD39 has an amino acid sequence selected from the group consisting of SEQ ID NOs: 1 to 21.

A27. The recombinant CD39 according to any one of the above embodiments, wherein the recombinant CD39 has the amino acid sequence of SEQ ID NO: 21.

A28. The recombinant CD39 according to any one of the above embodiments, wherein the recombinant CD39 is administered as monotherapy to said subject.

A29. The recombinant CD39 according to any one of the above embodiments, wherein the recombinant CD39 is co-administered with an additional therapeutic agent.

A30. The recombinant CD39 according to any one of the above embodiments, wherein the recombinant CD39 is co-administered with an additional therapeutic agent selected from a steroid, a metalloproteinase inhibitor, a serine protease inhibitor, a topical anesthetic emulsion, a spreading factor inhibitor, an anti-nausea agent, or an antibiotic.

A31. The recombinant CD39 according to any one of the above embodiments, wherein the AKI is SA-AKI.

A32. The recombinant CD39 according to any of the above embodiments, wherein the AKI is cardiac surgery associated AKI (CSA-AKI).

A33. The recombinant CD39 according to any of the above embodiments, wherein the recombinant CD39 is used in a method for preventing or treating one or more symptoms associated with AKI.

A34. A recombinant CD39 for use as a medicament, wherein the recombinant CD39 is to be administered at a dose of from about 0.1 mg/kg to about 10 mg/kg, for example at a dose of about 1 mg/kg, about 2 mg/kg, about 3 mg/kg, about 4 mg/kg or about 5 mg/kg.

A35. A recombinant CD39 for use in treating or preventing tissue damage, wherein the recombinant CD39 is to be administered at a dose of from about 0.1 mg/kg to about 10 mg/kg, for example at a dose of about 1 mg/kg, about 2 mg/kg, about 3 mg/kg, about 4 mg/kg or about 5 mg/kg.

A36. A recombinant CD39 for use in treating or preventing tissue damage, wherein the tissue damage is acute brain injury (stroke); acute multi-organ failure; delayed graft function after transplantation of kidney or other solid organs; burn damage; radiation damage; acute damage due to trauma and/or hypoxia, such as acute respiratory distress syndrome (ARDS) or lung injury; acute kidney injury, such as acute kidney injury secondary to thoracic surgery (e.g. aortic valve replacement, coronary artery bypass surgery) or sepsis or rhabdomyolysis or toxic effects of antibiotics or other medication; acute myocardial injury,

and wherein the recombinant CD39 is to be administered at a dose of from about 0.1 mg/kg to about 10 mg/kg, for example at a dose of about 1 mg/kg, about 2 mg/kg, about 3 mg/kg, about 4 mg/kg or about 5 mg/kg.

A37. A recombinant CD39 for use in the treatment of delayed graft function (DGF), acute respiratory distress syndrome (ARDS), acute myocardial infarction (AMI), traumatic brain injury (TBI)/acute ischemic stroke (AIS), ischemia-reperfusion injury (IRI), or combinations thereof often referred to as multi-organ failures (MOF),

and wherein the recombinant CD39 is to be administered at a dose of from about 0.1 mg/kg to about 10 mg/kg, for example at a dose of about 1 mg/kg, about 2 mg/kg, about 3 mg/kg, about 4 mg/kg or about 5 mg/kg.

A38. A recombinant CD39 for use according to embodiments A36 or A37, wherein the recombinant CD39 is to be administered at a dose of about 1 mg/kg of the recombinant CD39.

A39. A recombinant CD39 for use according to embodiments A36 or A37, wherein the recombinant CD39 is to be administered at a dose of about 2 mg/kg of the recombinant CD39.

A40. A recombinant CD39 for use according to embodiments A36 or A37, wherein the recombinant CD39 is to be administered at a dose of about 4 mg/kg of the recombinant CD39.

A50. A recombinant CD39 for use according to embodiments A36 or A37, wherein the recombinant CD39 is to be administered at a dose of about 5 mg/kg of the recombinant CD39.

A51. A recombinant CD39 for use according to embodiments A36 or A37, wherein the recombinant CD39 is to be administered IV, e.g. is to be administered IV during about 2 hours.

A52. A recombinant CD39 according to any of the above embodiments, wherein the recombinant CD39 comprises the amino acid sequence of SEQ ID NO: 2

B1. A method of treating or preventing AKI in a subject, especially a subject in need thereof, said method comprises administering an recombinant CD39, typically in a therapeutically effective amount.

B2. The method according to embodiment B1, wherein the AKI is severe AKI.

B3. The method according to embodiment B1, wherein at least one symptom of AKI in a subject is reduced or abolished.

B.4. The method according to any one of the above embodiments B1-B3, wherein the severity of AKI is reduced by at least one stage, as defined by modified AKI Network (AKIN) criteria for serum creatinine.

B5. The method according to any one of embodiments B1-B4, wherein the risk of AKI is reduced.

B6. The method according to any one of embodiments B1-B4, wherein the severity of AKI is reduced.

B7. The method according to any one of the above embodiments B1-B4, wherein the incidence of acute kidney disease severity is reduced, evaluated by renal function and Major Adverse Kidney Events (MAKE).

B8. The method according to any one of the above embodiments B1-B4, wherein the progression of AKI, e.g. sepsis-AKI, is prevented.

B9. The method according to any one of the above embodiments B1-B8, wherein one or more major adverse kidney events are improved, e.g. renal impairment is decreased.

B10. A method of treating or preventing sepsis in a subject, especially a subject in need thereof, said method comprises administering an recombinant CD39, typically in a therapeutically effective amount.

B11. The method according to embodiment B10, wherein diagnosis of sepsis is performed according to criteria defined by The Third International Consensus.

B12. The method according to embodiment B10 or B11, wherein sepsis and septic Shock (Sepsis-3) are defined based on:
Suspected or confirmed infection, AND
Acute increase of sequential organ failure score (SOFA) score of 2 or more (excluding renal component). The baseline SOFA score should be assumed to be zero unless the participant is known to have pre-existing (acute or chronic) organ dysfunction before the onset of infection.

B13. The method according to any one of the above embodiments B1-B12, wherein the recombinant CD39 is to be administered at a dose of from about 0.1 mg/kg to about 10 mg/kg, for example at a dose of about 1 mg/kg, about 2 mg/kg, about 3 mg/kg, about 4 mg/kg or about 5 mg/kg.

B14. The method according to any one of the above embodiments B1-B13, wherein the recombinant CD39 is to be administered IV, e.g. is to be administered IV during about 2 hours.

B15. The method according to any one of the above embodiments B1-B14, wherein the recombinant CD39 is to be administered within a number of hours after AKI, or sepsis, is first diagnosed for said subject, e.g. within about 48 hours, or less, after AKI is first diagnosed for said subject (e.g., within 12 hours, within 24 hours, or within 36 hours, for example).

B16. The method according to any one of the above embodiments B1-B15, wherein the recombinant CD39 is to be administered to the subject daily, e.g. IV, at a dosing of about 4 or 5 mg/kg, by infusion, within a time interval initiating from suspected or confirmed AKI diagnosis, or sepsis diagnosis, for said subject.

B17. The method according to any one of the above embodiments B1-B16, wherein the recombinant CD39 has an amino acid sequence selected from the group consisting of SEQ ID NOs: 1 to 21.

B18. The method according to any one of the above embodiments B1-B17, wherein the recombinant CD39 has the amino acid sequence of SEQ ID NO: 21.

B19. The method according to any one of the above embodiments B1-B18, wherein the recombinant CD39 is administered as monotherapy to said subject.

B20. The method according to any one of the above embodiments B1-B18, wherein the recombinant CD39 is co-administered with an additional therapeutic agent.

B21. The method according to embodiment B20, wherein the recombinant CD39 is co-administered with an additional therapeutic agent selected from a steroid, a metalloproteinase inhibitor, a serine protease inhibitor, a topical anesthetic emulsion, a spreading factor inhibitor, an anti-nausea agent, or an antibiotic.

B22. The method according to any one of the above embodiments B1-B21, wherein the AKI is SA-AKI or cardiac surgery associated AKI (CSA-AKI).

B23. A method for treatment using a recombinant CD39, wherein the recombinant CD39 is to be administered at a dose of from about 0.1 mg/kg to about 10 mg/kg, for example at a dose of about 1 mg/kg, about 2 mg/kg, about 3 mg/kg, about 4 mg/kg or about 5 mg/kg.

B24. A method for treating or preventing tissue damage using a recombinant CD39, wherein the recombinant CD39 is to be administered at a dose of from about 0.1 mg/kg to about 10 mg/kg, for example at a dose of about 1 mg/kg, about 2 mg/kg, about 3 mg/kg, about 4 mg/kg or about 5 mg/kg.

B25. A method for treating or preventing tissue damage using a recombinant CD39, wherein the tissue damage is acute brain injury (stroke); acute multi-organ failure; delayed graft function after transplantation of kidney or other solid organs; burn damage; radiation damage; acute damage due to trauma and/or hypoxia, such as acute respiratory distress syndrome (ARDS) or lung injury; acute kidney injury, such as acute kidney injury secondary to thoracic surgery (e.g. aortic valve replacement, coronary artery bypass surgery) or sepsis or rhabdomyolysis or toxic effects of antibiotics or other medication; acute myocardial injury,

and wherein the recombinant CD39 is to be administered at a dose of from about 0.1 mg/kg to about 10 mg/kg, for example at a dose of about 1 mg/kg, about 2 mg/kg, about 3 mg/kg, about 4 mg/kg or about 5 mg/kg.

B26. A method for the treatment of delayed graft function (DGF), acute respiratory distress syndrome (ARDS), acute myocardial infarction (AMI), traumatic brain injury (TBI)/acute ischemic stroke (AIS), ischemia-reperfusion injury (IRI), or combinations thereof often referred to as multi-organ failures (MOF), using a recombinant CD39

and wherein the recombinant CD39 is to be administered at a dose of from about 0.1 mg/kg to about 10 mg/kg, for example at a dose of about 1 mg/kg, about 2 mg/kg, about 3 mg/kg, about 4 mg/kg or about 5 mg/kg.

B27. A method according to any of the above embodiments B1-B26, wherein the recombinant CD39 comprises the amino acid sequence of SEQ ID NO: 21.

### DEFINITIONS

The term “CD39” as used herein refers to the human protein CD39 (Cluster of Differentiation 39), also known as ectonucleoside triphosphate diphosphohydrolase-1 (NTPDase1 or Ecto-ATPDase 1). CD39 is an ectonucleotidase present on the cell surface with a catalytic site on the extracellular face that catalyzes the hydrolysis of γ- and β-phosphate residues of triphospho- and diphosphonucleosides to the monophosphonucleoside derivative. Human CD39 in particular has the amino acid sequence of SEQ ID NO: 22 and/or is represented by UniProt entry P49961.

A “variant of CD39” refers to a protein derived from human CD39 which has an amino acid sequence being at least 60% identical to human CD39 over the entire length of human CD39. In certain embodiments, the variant of CD39 only consists of the extracellular domain or a part thereof of human CD39 (amino acids 38 to 478 of SEQ ID NO: 22). In particular, the variant of CD39 has an amino acid sequence being at least 80% identical to the extracellular domain of human CD39 over the entire length of amino acid positions 38 to 478 of human CD39. In particular, the variant of CD39 is a soluble variant, i.e. a variant which lacks the transmembrane domains of human CD39 and its membrane anchors. In certain embodiments, the variant has an N terminal deletion of 30 to 50 amino acids, a C terminal deletion of 20 to 40 amino acids and/or a central deletion of 10 to 15 amino acids compared to human CD39. In addition, the variant may comprise up to 5 point mutations compared to human CD39. Certain variants of CD39 are disclosed in WO 2020/016804. In particular, the human recombinant CD39 variant has an amino acid sequence selected from the group consisting of SEQ ID NOs: 1 to 21.

The “recombinant CD39” refers in particular to the human recombinant CD39 variant having an amino acid sequence selected from the group consisting of SEQ ID NOs: 1 to 21.

The term “about” in relation to a numerical value x means, for example, +/−10%. When used in front of a numerical range or list of numbers, the term “about” applies to each number in the series, e.g., the phrase “about 1-5” should be interpreted as “about 1-about 5”, or, e.g., the phrase “about 1, 2, 3, 4” should be interpreted as “about 1, about 2, about 3, about 4, etc.”

The word “substantially” does not exclude “completely,” e.g., a composition which is “substantially free” from Y may be completely free from Y. Where necessary, the word “substantially” may be omitted from the definition of the disclosure.

As used herein, a subject is “in need of” a treatment if such subject would benefit biologically, medically or in quality of life from such treatment.

The term “pharmaceutically acceptable” means a nontoxic material that does not interfere with the effectiveness of the biological activity of the active ingredient(s).

The term “treatment” or “treat” is herein defined as the application or administration of apyrase according to the invention to a subject, or application or administration a pharmaceutical composition comprising said recombinant CD39 to a subject, or an isolated tissue or cell line from a subject, where the subject has tissue damage, a symptom associated with tissue damage, where the purpose is to alleviate, ameliorate, or improve the tissue damage or any associated symptoms of the tissue damage inter alia by reducing levels of extracellular ATP.

By “treatment” is also intended the application or administration of a pharmaceutical composition comprising an recombinant CD39 to a subject, or application or administration of a pharmaceutical composition comprising apyrase of the invention to an isolated tissue or cell line from a subject, where the subject has an tissue damage or a symptom associated with tissue damage, where the purpose is to alleviate, ameliorate, or improve the tissue damage or any associated symptoms of the tissue damage.

The term “prevent” or “preventing” refer to prophylactic or preventative treatment; it is concerned about delaying the onset of, or preventing the onset of the disease, disorders and/or symptoms associated thereto.

As used herein, the term “administration” or “administering” of the subject compound means providing a compound of the invention and prodrugs thereof to a subject in need of treatment. Administration “in combination with” one or more further therapeutic agents includes simultaneous (concurrent) and consecutive administration in any order, and in any route of administration.

As used herein, a “therapeutically effective dose” refers to a dose (an amount) of a recombinant CD39 that is effective, upon single or multiple dose administration to a patient (such as a human) for treating, preventing, preventing the onset of, curing, delaying, reducing the severity of, ameliorating at least one symptom of a disorder or recurring disorder, or prolonging the survival of the patient beyond that expected in the absence of such treatment. When applied to an individual active ingredient (e.g., recombinant CD39) administered alone, the term refers to that ingredient alone. When applied to a combination, the term refers to combined doses or amounts of the active ingredients that result in the therapeutic effect, whether administered in combination, serially or simultaneously.

The phrase “dosage regimen” means the regimen used to treat an illness, e.g., the dosing protocol used during the treatment of AKI, SA-AKI or CSA-AKI.

The phrase “means for administering” is used to indicate any available implement for systemically administering a drug to a patient, including, but not limited to, a pre-filled syringe, a vial and syringe, an injection pen, an autoinjector, an intravenous (i.v.) drip and bag, a pump, a patch pump, etc. With such items, a patient may self-administer the drug (i.e., administer the drug on their own behalf) or a physician may administer the drug.

Determination of AKI, e.g. severe AKI, will be grounded in the staging system outlined in the KDIGO Guidelines for acute kidney injury. This classification system stages AKI into three levels based on the extent of sCr elevation in parallel with the degree and duration of oliguria; patients are classified based on the worst finding (either sCr or oliguria). Three stages of AKI are classified in the table below:

TABLE 1
Staging of AKI

| Stage | Serum creatinine | Urine output |
|---|---|---|
| 1 | 1.5-1.9 times baseline | <0.5 ml/kg/h for 6-12 hours |
| | OR | |
| | ≥0.3 mg/dl (≥26.5 μmol/l) increase | |
| 2 | 2.0-2.9 times baseline | <0.5 ml/kg/h for ≥12 hours |
| 3 | 3.0 times baseline | <0.3 ml/kg/h for ≥24 hours |
| | OR | OR |
| | Increase in serum creatinine | Anuria for ≥12 hours |
| | to ≥4.0 mg/dl (≥353.6 μmol/l) | |
| | OR | |
| | Initiation of renal replacement | |
| | therapy | |

The diagnosis of sepsis can be done for example according to the criteria defined by The Third International Consensus Definition for Sepsis and Septic Shock (Sepsis-3), with an additional diagnosis of AKI (stage 1 or greater using the KDIGO serum creatinine-based criteria).

The term “cardiac surgery” is used to refer to non-emergent open chest cavity major cardiopulmonary bypass (CPB) surgery with expected CPB time>1 hour, defined be any of the following options:
a. combined coronary artery bypass grafting (CABG) surgery and surgery of one or more cardiac valve (valve(s) surgery)
b. surgery of more than one cardiac valve (valve surgery)
c. surgery of the aortic root or ascending part of the aorta, or in combination with the aortic valve
d. aortic root or ascending part of the aorta, combined with CABG and/or valve(s) surgery.

“Sequential Organ Failure Assessment Score”: The SOFA (sequential organ failure assessment) score was developed to assess the acute morbidity of critical illness at a population level and has been widely validated as a tool for this purpose across a range of healthcare settings and environments. Following the development of new definitions, it is now used as a key criterion in the diagnosis of sepsis syndrome on an individual patient level. The score is routinely calculated on admission to ICU and at each 24-hour period that follows. It is composed of six criteria which reflect the function of a specific organ system (respiratory, cardiovascular, renal, neurological, hepatic and hematological) and allocates a score of 0-4.

Scores ranges from 0-24, with higher scores indicating greater dysfunction.

The SOFA score has been applied in a range of clinical and research applications.

In the context of randomized controlled trials, a meta-analysis identified 25 studies where the change in SOFA score from baseline or maximum to a defined time point was used and revealed a strong association between change in SOFA and mortality—with 32% of the observed mortality effects explained by the delta SOFA score. This association of SOFA score at admission and during ICU stay with longer term outcomes has led to the acceptance by the EMA that use of delta SOFA is a valid surrogate endpoint in exploratory clinical trials of participants with sepsis.

“Acute Physiology and Chronic Health Evaluation”: The APACHE-II (Acute Physiology and Chronic Health Evaluation II) score is a severity-of-disease classification system for the assessment of critically unwell patients. It is applied within 24 hours of admission of a patient to an ICU or intermediate/HDU. An integer score from 0-71 is computed based on several measurements: higher scores correspond to more severe disease and a higher risk of death in ICU patients.

“Renal replacement therapy” (RRT): Meeting at least one criterion of the below, makes initiation of RRT recommended:
1. Anuria (negligible urine output for 6 hours)
2. Severe oliguria (urine output<200 mL over 12 hours)
3. Hyperkalemia (potassium concentrations>6.5 mmol/L)
4. Severe metabolic acidosis (pH<7.2 despite normal or PaCO2 low partial pressure of carbon dioxide in arterial blood)
5. Volume overload
6. Pronounced azotemia (urea concentrations>30 mmol/L (>84 mg/dL) or creatinine concentrations>300 μmol/L (>3.4 mg/dL))
7. Clinical complications of uremia (e.g., encephalopathy, pericarditis, neuropathy)

“Major Adverse Kidney Events” (MAKE) is a composite endpoint including death, need for RRT and worsening renal function (eGFR≥25% reduction from reference baseline). It can be assessed at defined intervals/timepoints and is increasingly being recognized as an important participant-centered outcome from studies in participants who are critically unwell. incidence of MAKE will be calculated at 30, 60 and 90 days. The number of participants with the composite endpoint MAKE, where MAKE is defined as participants with death, RRT, or ≥25% reduction in eGFR from reference baseline. eGFR is calculated using CKD-EPI formula without race factor.

In an additional preferred embodiment the disclosure relates to the use recombinant CD39 according to the invention for the treatment of sepsis associated acute kidney injury, SA-AKI.

In an additional preferred embodiment the disclosure relates to the use recombinant CD39 according to the invention for the treatment of cardiac surgery associated acute kidney injury, CSA-AKI.

Provided below are exemplary, non-limiting embodiments of the methods described in this disclosure.

### EXAMPLES

### Example 1: Therapeutic Compositions

Therapeutic proteins are typically formulated either in aqueous form ready for administration or as lyophilizate for reconstitution with a suitable diluent prior to administration. A protein may be formulated either as a lyophilizate, or as an aqueous composition, for example in pre-filled syringes.

Suitable formulation can provide an aqueous pharmaceutical composition or a lyophilizate which can be reconstituted to give a solution with a high concentration of the therapeutic protein active ingredient and a low level of protein aggregation for delivery to a patient. High concentrations of protein are useful as they reduce the amount of material which must be delivered to a patient (the dose). Reduced dosing volumes minimize the time taken to deliver a fixed dose to the patient. The aqueous compositions of the invention with high concentration of proteins are particularly suitable for subcutaneous administration.

Thus the invention provides an aqueous pharmaceutical composition, suitable for administration in a subject, e.g., for subcutaneous administration, comprising a therapeutic protein.

The therapeutic protein may be used as a pharmaceutical composition when combined with a pharmaceutically acceptable carrier. Such a composition may contain, in addition to a therapeutic protein, carriers, various diluents, fillers, salts, buffers, stabilizers, solubilizers, and other materials well known in the art. The characteristics of the carrier will depend on the route of administration. The pharmaceutical compositions for use in the disclosed methods may also contain additional therapeutic agents for treatment of the particular targeted disorder.

Because the indications for use according to the present invention (e.g., treatment of SA-AKI) require immediate treatment, rather than oral administration and may be given as an IV or liquid formulation.

A recombinant CD39 (in the following “CD39*”) having the amino acid sequence of SEQ ID NO: 21, is an engineered, highly effective, soluble, and stabilized human recombinant CD39 enzyme. The enzyme hydrolyzes extracellular ATP and ADP to adenosine monophosphate (AMP), which is a rate limiting step in conversion of extracellular ATP to adenosine. AMP is further catalyzed by the ecto-5′-nucleotidase CD73 to adenosine. The enzymatic activities of CD39 and CD73 play strategic roles in calibrating the duration, magnitude, and chemical nature of purinergic signals delivered to immune cells, driving a shift from an ATP-driven pro-inflammatory environment to an anti-inflammatory milieu induced by adenosine. It is becoming increasingly appreciated that rebalancing this equilibrium could change the course and outcome of several pathophysiological events, such as SA-AKI.

Exemplary methods of obtaining certain variants of CD39, recombinant CD39, such as CD39*, are disclosed in WO2020/016804.

### Example 2. Assessment in Non-Clinical Studies

CD39* has been investigated extensively in preclinical models. In human whole blood CD39* dose dependently inhibited ATP/Lipopolysaccharide (LPS)-induced IL-1ß secretion as well as ADP induced platelet aggregation. CD39* has been proven beneficial in preclinical models of acute organ injuries, including mouse models of ischemia-reperfusion induced AKI where CD39* was dose dependently preserving kidney function. The protection could be shown in terms of kidney structure, kidney inflammation, and acute tubular necrosis, which are also hallmarks of human AKI pathophysiology (results not shown).

In addition, the common biomarkers of kidney injury, function and repair in the kidney tissue and in the urine of animal undergoing AKI normalized to normal levels with CD39* treatment.

Biomarkers of target engagement and proximal pharmacodynamic extracellular ATP and ADP in the urine of animals after AKI are dose-dependently reduced and extracellular AMP and adenosine are dose-dependently increased.

The in vitro potency for CD39* in whole blood (WB) assays (LPS/ATP induced IL-1B release and ADP-induced platelet aggregation) was demonstrated to be comparable for rat, minipig and human. The similarity in pharmacology across species allowed to predict and explore an exposure range expected to be safe in light of animal safety and pharmacology data and reaching expected therapeutic doses. These PD whole blood assays revealed in-vitro IC90s of approximately 1.66 to 2.66 μg/mL for human.

### Example 3. Phase I, Randomized, Placebo-Controlled, Participant-Blinded Study in Healthy Volunteers

This study is a Phase I, randomized, placebo-controlled, participant-blinded study in healthy volunteers. In this study, a single IV. dose of CD39* (0.1, 0.3, 1.0 and 2.0 mg/kg, and 4.0 mg/kg) have been administered as a 2-hour infusion to healthy participants.

The administration of 0.1, 0.3, 1.0 and 2.0 mg/kg, and 4.0 mg/kg of CD39* or placebo as a single IV infusion was well tolerated and did not reveal any safety signals related to the investigational medicinal product (IMP).

CD39* was safe and well tolerated without any immunogenicity findings.

The PK results are fully in line with previously predicted CD39* serum concentrations.

The pathway engagement markers demonstrate dose-dependent and reversible inhibition of (i) ex vivo ADP-induced platelet aggregation and (ii) ex vivo LPS/ATP-induced IL-1ß release in CD39* treated subjects resulting in ex vivo IC90s of 0.25 and 7.8 μg/mL for inhibition of platelet aggregation and IL-1ß release, respectively.

CD39* at doses higher than 0.1 mg/kg induced a dose dependent and reversible reduction of free inorganic pyrophosphate (PPi) levels with maximum effect reached within hours post dose.

Exposure determined by Cmax and AUCinf demonstrated dose proportional behavior for this dose range. A 3—, 10- and 20-fold increase of dose when ascending from 0.1 mg/kg to 0.3 mg/kg, 1.0 mg/kg and 2.0 mg/kg, respectively, resulted in a 3—, 12- and 24-fold increase of average Cmax and a 3—, 11- and 17-fold increase of average AUCinf.

Dose proportionality was also demonstrated by assessing correlation between dose and Cmax and between dose and AUC via a power model.

CD39* exposure was investigated throughout SAD Cohorts 1 to 4 (0.1, 0.3, 1.0 and 2.0 mg/kg IV) in human skin by quantifying CD39* in interstitial fluid collected via suction blisters at 24 h and 168 h post-dose. CD39* exposure in interstitial fluid increased in a fairly dose proportional manner. By increasing the dose 3-, 10- and 20-fold, mean interstitial fluid concentrations increased 3-, 10 and 33-fold at 24 hours post-dose and 3-, 9- and 18-fold at 168 hours post-dose, respectively.

The study results showed that CD39* was safe and well tolerated.

### Example 4. A Method of Treating Sepsis Associated Acute Kidney Injury Comprising Administering a Therapeutically Effective Dose of Recombinant CD39* to a Subject in Need of Such Treatment

This is a multicenter, participant and investigator-blinded, randomized, placebo-controlled study to characterize PK/PD profile and to evaluate the safety and the efficacy of human recombinant CD39 enzyme CD39* in hospitalized adult participants with diagnosis of sepsis and acute kidney injury (AKI). Approximately 20 participants will be randomized in the study. The study consists of a screening period (up to 48 hours), treatment period (Day 1), and post-treatment period (Day 2 to 90).

Screening will take place during hospitalization in intensive care unit (ICU) or intermediate/high dependency unit (HDU) where potential participants will undergo screening to assess the presence of sepsis and AKI based on sequential organ failure assessment (SOFA) and kidney disease improving global outcomes (KDIGO) scores.

Participants will be randomly assigned to receive either CD39* at 2 mg/kg or placebo using a 3:1 allocation ratio (CD39*: placebo). A single dose of the study treatment will be administered via 2-hour IV at the randomization visit in a participant and investigator-blinded fashion. The study treatment must be administered within 48 hours, after SA-AKI is first diagnosed, but ideally as soon as possible. Two PK samples will be taken at Day 1, one prior to study treatment and one immediately after the completion of IV infusion (within 15 minutes), and additional samples will be taken at Day 2, 3, 5, 8, 14, 30, 60 and 90, optimally always at the same time of the day as when dosing of CD39* started on Day 1. All participants will be followed-up for a total duration of 90 days.

Safety will be monitored during the entire course of the study. All adverse event (AE) and serious adverse event (SAE) will be collected and reported following standard processes and local regulations.

At 2 mg/kg, the expected exposure systemically and in relevant tissue interstitia is believed to inhibit purinergic signaling and provide a beneficial pharmacological effect. Total exposure (AUC_{inf}) of a single dose of 2 mg/kg in human is approximately 10.4-fold lower compared to the NOAEL overall exposure from 4 IV doses of 10 mg/kg CD39* administered q4d in the 2 weeks minipig study (compared to AUC_{tau,ss} from the 4th dose in minipig at steady state the exposure margin is 2.6-fold) and Cₘₐₓ shows a 7-fold margin to the NOAEL exposure. Participants will be followed-up for a total duration of 90 days.

Participants eligible for inclusion in this study must meet all of the following criteria at screening assessment, unless otherwise specified at randomization (Day 1):
1. Signed informed consent must be obtained prior to participation in the study.
2. ≥18 and ≤85 years of age.
3. Admitted to ICU or intermediate/HDU.
4. Diagnosis of sepsis according to criteria defined by The Third International Consensus Definitions for Sepsis and Septic Shock (Sepsis-3) based on:
   Suspected or confirmed infection
   SOFA score of 2 or more (excluding renal component)
5. Diagnosis of AKI Stage 1 or greater per the following criterion at randomization:
   An absolute increase in serum or plasma creatinine (CR) by ≥0.3 mg/dL (≥26.5 μmol/L) within 48 hours or presumed to have occurred in the previous 48 hours as compared to the reference creatinine baseline
   For hospital-acquired AKI, a stable serum creatinine obtained in the hospital prior to AKI should be used as reference baseline, otherwise, baseline serum creatinine in the following order of preference:
      1. Median value within 3 months of the hospital admission. If not available:
      2. Median value between 3 and 6 months prior to hospital admission. If not available:
      3. At hospital admission.

Participants meeting any of the following criteria at screening assessment, unless otherwise specified at randomization (Day 1), are not eligible for inclusion in this study.
1. Not expected to survive for 24 hours.
2. Not expected to survive for 30 days due to medical conditions other than SA-AKI.
3. History of CKD with a documented estimated GFR<45 ml/min prior to development of AKI.
4. Receiving RRT or a decision has been made to initiate RRT within 24 hours of admission.
5. Weight is less than 40 kg or more than 125 kg.
6. Has life support limitations (e.g., do not resuscitate, do not dialyze, do not intubate).
7. AKI diagnosis according to the AKI inclusion criteria for a period longer than 48 hours prior to study drug administration.
8. Presence of AKI for a period longer than 48 hours prior to study drug administration as suggested by clinical manifestations, e.g., prolonged oliguria or severe renal dysfunction (e.g., serum creatinine>4 mg/dL) on admission without a history of CKD.
9. Evidence of recovery from AKI based on the investigator's clinical judgement prior to randomization.
10. AKI is most likely attributable to causes other than sepsis such as nephrotoxic drugs (Non-steroidal anti-inflammatory drugs (NSAIDs), contrast, aminoglycosides), other medical conditions (e.g. heart failure, liver failure, acute abdominal aortic aneurysm, dissection, renal artery stenosis) or urinary obstruction.
11. Documented (biopsy proven) or suspected history of acute or sub-acute kidney diseases such as rapidly progressive glomerular nephritis (RPGN) and acute interstitial nephritis (AIN).
12. Patients who are post-nephrectomy.
13. Patients who are on dual antiplatelet therapy.
14. Patients who are thrombocytopeniat screening (Platelet count<100,000 microliter) or other high risk for bleeding in the opinion of the investigator.
15. Immunosuppressed patients:
   History of immunodeficiency diseases or known HIV test positive.
   Is receiving immunosuppressant treatment or is on chronic high doses (high-dose therapy exceeding 2 weeks of treatment) of steroids equivalent to prednisone/prednisolone 0.5 mg/kg/day, including solid organ transplant patients. Patients with septic shock treated with hydrocortisone (e.g., 3×100 mg) can be included.
16. Active hepatitis (defined as (a) abnormal liver enzymes (Alanine aminotransferase (ALT), Gamma-glutamyl transferase (GGT), ALP>3× Upper Limit of Normal (ULN) or (b)) for active hepatitis B or C infection, a positive Hepatitis B Virus (HBV) or Hepatitis C Virus (HCV) serology or patients with advanced chronic liver disease, confirmed by a Child-Pugh score of 10-15 (Class C).
17. Acute pancreatitis with no established source of infection.
18. Active hematological malignancy (previous hematological malignancies that are not actively treated are allowable).
19. Burns requiring ICU treatment.
20. Sepsis attributed to confirmed COVID-19.
21. Use of other investigational drugs within 5 half-lives of enrollment within 30 days (e.g., small molecules)/or until the expected pharmacodynamic effect has returned to baseline (e.g., biologics), whichever is longer; or longer if required by local regulations.
22. History of hypersensitivity to the study treatment or its excipients or to drugs of similar chemical classes.
23. Any medical conditions that could significantly increase risk of participants' safety by participating in this study according to investigator's judgement.
24. Women with a positive pregnancy test, pregnancy or breast feeding.
25. Women of child-bearing potential, defined as all women physiologically capable of becoming pregnant, unless they are using highly effective methods of contraception while taking study treatment and for 38 days of study treatment after stopping medication. Highly effective contraception methods include:
   Total abstinence (when this is in line with the preferred and usual lifestyle of the participant. Periodic abstinence (e.g. calendar, ovulation, symptothermal, post-ovulation methods) and withdrawal are not acceptable methods of contraception
   Female sterilization (have had surgical bilateral oophorectomy with or without hysterectomy), total hysterectomy, or bilateral tubal ligation at least six weeks before taking investigational drug. In case of oophorectomy alone, only when the reproductive status of the woman has been confirmed by follow-up hormone level assessment
   Male sterilization (at least 6 months prior to screening). For female participants on the study, the vasectomized male partner should be the sole partner for that participant
   Use of oral (estrogen and progesterone), injected, or implanted hormonal methods of contraception or placement of an intrauterine device (IUD) or intrauterine system (IUS), or other forms of hormonal contraception that have comparable efficacy (failure rate<1%), for example hormone vaginal ring or transdermal hormone contraception.

Study treatment drug will be administered as an 2 hour IV infusion (approximately) via a perfusor syringe at the clinical site by the study personnel in accordance with the specified study procedures.

### Example 5. a Randomized, Multi-Centric, Placebo-Controlled, Participant and Investigator-Blinded Study to Evaluate the Safety, Tolerability and Efficacy of Single i.v. Infusion of CD39* in Adult Patients at Risk for Acute Kidney Injury Following Cardiac Surgery

This is a non-confirmatory, randomized, multi-centric, placebo-controlled, participant and investigator-blinded study. Approximatively 120 adult patients at risk for acute kidney injury following cardiac surgery will be enrolled in the study.

There will be 2 groups in the study:
Placebo
CD39*4 mg/kg

Participants enrolling into this study will be managed according to current medical and surgical standard of care and as per standard institutional procedures. The study will consist of a pre-operative period (screening visit), a treatment period (Day 1) and a follow-up period (Day 2 to Day 90). Participants will be followed up on Day 2, 3, 4, 5, 6 and Day 8 in hospital (or at home/in rehab until if discharged earlier than Day 8), and then as an out-patient until the end of study (Day 30 and 90 visits).

On Day 1, the participant will undergo cardiac surgery. The randomization will occur once the cardiopulmonary bypass time is confirmed to be equal or higher than 60 min. Participants will be stratified according to the following factor: eGFR>=30 mL/min/1.73 m2 vs eGFR<30 mL/min/1.73 m2. Once the wound is closed and it has been confirmed that there is no ongoing bleeding, the participant will receive a single dose of CD39* or placebo. The administration should start as soon as possible after wound closure and no later than 60 min after wound closure.

In this study, patients will be administered the study drug at a dose of 4 mg/kg or placebo via a 2-hour intravenous infusion in a double-blinded fashion. The dose of 4 mg/kg is the highest safe dose tested in healthy volunteers and the expected exposure systemically and in relevant tissue interstitia is thought to inhibit purinergic signalling and provide a beneficial pharmacological effect. Total exposure (AUCinf) of a single dose of 4 mg/kg in human is approximately 5.2-fold lower compared to the NOAEL overall exposure from 4 i.v. doses of 10 mg/kg CD39* administered q4d in the 2 weeks minipig study (compared to AUC, from the 4th dose in minipig the exposure margin is 1.3-fold) and Cmax shows a 3.5-fold margin to the NOAEL exposure. Patients will be followed up for a total duration of 90 days.

Participants eligible for inclusion in this study must meet all of the following criteria:
1. Signed informed consent must be obtained prior to participation in the study.
2. Participants must be able to communicate well with the investigator and to understand and comply with the requirements of the study.
3. Male and female patients ≥45 years at screening.
4. Participants must weigh at least 50 kg and maximum 150 kg to participate in the study and must have a body mass index (BMI) below 40. BMI=Body weight (kg)/[Height (m)]²
5. At screening, vital signs (systolic and diastolic blood pressure and pulse rate) will be assessed in the sitting position. Sitting vital signs should be within the following ranges:
   a. oral body temperature between 35.0-37.5 °C.
   b. blood pressure (systolic 100-160 mmHg, diastolic <100 mmHg)
   c. pulse rate (50-100/min) stable with or without medication as per Investigator assessment. If vital signs are outside these ranges, the Investigator may obtain two additional readings, so that up to three consecutive assessments are made. At least the last reading must be within the ranges provided above in order for the participant to qualify.
6. No known increase in SCr of ≥25% at screening visit compared to a previous value not older than 6 weeks as documented by the site's local laboratory using standard assay methodology.
7. eGFR <60 mL/min/1.73m2 (CKD Stage 3A or higher) at screening (calculated using Chronic Kidney Disease Epidemiology Collaboration (CKD-EPI 2021) equation).
8. Non-emergent open chest cavity major cardiopulmonary bypass (CPB) surgery with expected CPB time >1 hour, defined by any of the following options:
   a. combined coronary artery bypass grafting (CABG) surgery and surgery of one or more cardiac valve (valve(s) surgery).
   b. surgery of more than one cardiac valve (valve surgery).
   c. surgery of the aortic root or ascending part of the aorta, or in combination with the aortic valve.
   d. aortic root or ascending part of the aorta, combined with CABG and/or valve(s) surgery.
   Note 1: If CABG alone or single valve surgery, participants are required to have at least one additional risk factor for AKI:
   i. medication-dependent diabetes mellitus defined as Type 1 or Type 2 prior to hospitalization for cardiac surgery.
   ii. albuminuria defined as urine albumin-to-creatinine ratio >300 mg/g (30 mg/mmol) or urinary albumin excretion >300 mg/24 h, at screening.
   iii. age ≥70 years old at screening.
   iv. history of congestive heart failure requiring hospitalization.
   Note 2: Minimally invasive cardiac surgeries (MICS) as performed through small thoracotomies meet the definition of open chest cavity interventions and patients undergoing MICS can be enrolled if CPB time >1 hour and inclusion criteria #8 are met.
   Note 3: Transcatheter aortic valve implantation (TAVI) or transcatheter aortic valve replacement (TAVR) are allowed only if performed in combination with surgeries defined under inclusion criterion 8.

Participants meeting any of the following criteria are not eligible for inclusion in this study.
1. Have an eGFR at screening <15 mL/min/1.73 m2 (calculated using CKD-EPI 2021 equation).
2. Currently receiving renal replacement therapy.
3. Patients with bleeding risk at screening, defined as:
   History of bleeding with suspected or confirmed bleeding disorder or any other high risk for bleeding in the opinion of the investigator
   Thrombocytopenia: platelet count<100×10⁹/L
   Platelet less functional: ADP induced platelet aggregation lower than 60%
   Pre-existing coagulation factor deficiency: including, but not limited to fibrinogen <2.5-2.8 g/L
4. Any emergency surgeries performed less than 30 days before screening, including aortic dissection, and/or major congenital heart defects.
5. Scheduled to undergo cardiac surgery off CPB or with hypothermic circulatory arrest.
6. Scheduled to undergo TAVI or TAVR only or single vessel, minimally invasive direct coronary artery bypass (MIDCAB) off-pump surgeries or left ventricular assist device (LVAD) implantation (Note: See Inclusion Criteria 8).
7. Cardiogenic shock or hemodynamic instability within four weeks prior to surgery, requiring inotropes or vasopressors or mechanical devices such as intra-aortic balloon counter-pulsation (IABP).
8. Have required any of the following within four weeks prior to cardiac surgery: defibrillator, mechanical ventilation, IABP, LVAD, other forms of mechanical circulatory support (MCS).
9. Have received cardiopulmonary resuscitation (CPR) within 30 days prior to cardiac surgery.
10. Recent stent implantation≤30 days prior to cardiac surgery or other conditions necessitating restart of P2Y12 inhibitor therapy within 48 hours or less after surgery.
11. Duration of cardiopulmonary bypass (CPB)<60 minutes.
12. Patient with active bleeding at the end of the surgery as per assessment of surgical team.
13. Use of other investigational drugs at the time of enrollment, or within 5 half-lives of enrollment, or until the expected PD effect has returned to baseline, whichever is longer; or longer if required by local regulations.
14. History of hypersensitivity to any of the study treatments or its excipients or to drugs of similar chemical classes.
15. Known history (within 6 months prior to screening) or current clinically significant arrhythmias associated with syncope, dyspnea or hemodynamic instability.
16. Patients who are post-nephrectomy.
17. Concomitant use of agents known to prolong the QT interval unless they can be permanently discontinued for the duration of study (QT>500 ms and at the discretion of the investigator).
18. Taking medications prohibited by the protocol.
19. History of malignancy of any organ system (other than localized basal cell carcinoma of the skin or in-situ cervical cancer), treated or untreated, within the past 5 years, regardless of whether there is evidence of local recurrence or metastases.
20. Donation or loss of >450 mL of blood or >200 ml of Plasma within four weeks prior to dosing, or longer if required by local regulation.
21. Have a history of any organ or cellular transplant which requires active immunosuppressive treatment which can interfere with kidney function (e.g., calcineurin inhibitors).
22. Have ongoing sepsis or history of sepsis within the past 8 weeks or untreated diagnosed infection prior to screening visit. Sepsis is defined as presence of a confirmed pathogen, along with fever or hypothermia, and hypoperfusion or hypotension.
23. Heart failure stage IV as defined by the New York Heart Association (NYHA) Functional Classification.
24. Left ventricular ejection fraction of <30% as per most recent echocardiography available.
25. Recent (within the last three years) and/or recurrent history of autonomic dysfunction (e.g., recurrent episodes of fainting, palpitations, etc.).
26. Any surgical or medical condition which might significantly alter the absorption, distribution, metabolism, or excretion of drugs, or which may jeopardize the participant in case of participation in the study. The Investigator should make this determination in consideration of the participant's medical history and/or clinical or laboratory evidence of any of the following:
   Inflammatory bowel disease, peptic ulcers, gastrointestinal including rectal bleeding;
   Major gastrointestinal tract surgery such as gastrectomy, gastroenterostomy, or bowel resection;
   Pancreatic injury or pancreatitis;
   Liver disease or liver injury as indicated by abnormal liver function tests: ALT (SGPT), AST (SGOT), GGT, alkaline phosphatase and serum bilirubin will be tested.
   Evidence of urinary obstruction or difficulty in voiding at screening.
   If necessary, laboratory testing may be repeated on one occasion (as soon as possible) prior to randomization, to rule out any laboratory error.
27. History of immunodeficiency diseases, (evaluated as per local testing??) Add Tuberculosis as well?
28. (History) of Chronic infection with Hepatitis B (HBV) or Hepatitis C (HCV). A positive HBV surface antigen (HBsAg) test, or if standard local practice, a positive HBV core antibody test, excludes a participant.
   Active hepatitis defined by active AST/ALT??? (from sepsis trial?)
29. History of drug abuse or unhealthy alcohol use^{#} within the 12 months prior to dosing, or evidence of such abuse as indicated by the laboratory assays conducted during screening. ^{#}Unhealthy alcohol use is defined as a history of, or current alcohol misuse/abuse, defined as “Five or more drinks on the same occasion on each of 5 or more days in the past 30 days.”
30. Pregnant or nursing (lactating) women.
31. Women of child-bearing potential, defined as all women physiologically capable of becoming pregnant, unless they are using highly effective methods of contraception while taking study treatment and until the end of study. Highly effective contraception methods include:
   Total abstinence (when this is in line with the preferred and usual lifestyle of the participant. Periodic abstinence (e.g. calendar, ovulation, symptothermal, post-ovulation methods) and withdrawal are not acceptable methods of contraception.
   Female bilateral tubal ligation, female sterilization (have had surgical bilateral oophorectomy with or without hysterectomy) or total hysterectomy at least six weeks before taking study treatment. In case of oophorectomy alone, only when the reproductive status of the woman has been confirmed by follow up hormone level assessment.
   Male sterilization (at least 6 months prior to screening). For female participants on the study, the vasectomized male partner should be the sole partner for that participant.
   Use of oral (estrogen and progesterone), injected, or implanted hormonal methods of contraception or placement of an intrauterine device (IUD) or intrauterine system (IUS), or other forms of hormonal contraception that have comparable efficacy (failure rate <1%), for example hormone vaginal ring or transdermal hormone contraception.

### Example 6 a Multicenter, Randomized, Double-Blind, Placebo-Controlled, Four-Arm, Parallel-Group, Dose-Finding Phase 2b Study to Investigate the Safety and Efficacy of CD39* in the Treatment of Patients with Sepsis-Associated Acute Kidney Injury (SA-AKI)

This is a double-blind, placebo-controlled, four-arm, parallel-group, dose-finding phase 2b study. Approximately 320 participants will be randomized in a 3:1:1:3 ratio to one of three single CD39* doses of 4 mg/kg, 2 mg/kg, 1 mg/kg or to placebo respectively.

### Key Inclusion Criteria

≥18 to ≤85 years of age
Admitted to ICU or intermediate care unit/high dependency care unit (HDU)
Diagnosis of sepsis according to criteria defined by The Third International Consensus Definitions for Sepsis and Septic Shock (Sepsis-3) based on:
   Suspected or confirmed infection AND
   Acute increase of SOFA score of 2 or more (excluding renal component). The baseline SOFA score should be assumed to be zero unless the participant is known to have pre-existing (acute or chronic) organ dysfunction before the onset of infection
Diagnosis of AKI Stage 1 or greater per the following criterion at randomization: An absolute increase in serum or plasma creatinine by ≥0.3 mg/dl (≥26.5 μmol/L) within 48 hours or presumed to have occurred in the previous 48 hours as compared to the reference pre-sepsis creatinine.
   For hospital-acquired AKI, a stable serum creatinine obtained in the hospital prior to AKI diagnosis should be used as the reference serum creatinine.
   For patients presenting from community, the reference pre-sepsis serum creatinine should be estimated using the following order of preference:
   1. The most recent value within 3 months of the hospital admission. If not available:
   2. The most recent value between 3 and 12 months prior to hospital admission. If not available:
   3. At hospital admission

### Key Exclusion Criteria

History of chronic kidney disease (CKD) with a documented eGFR <45 mL/min prior to admission to hospital.
eGFR<45 mL/min at admission without any other reference serum eGFR within last 12-months
Receiving renal replacement therapy (RRT) or a decision has been made to initiate RRT within 24 hours of admission
Sepsis diagnosis according to sepsis inclusion criteria for a period longer than 72 hours prior to ICU admission
AKI diagnosis according to AKI inclusion criteria over 48 hours after admission to ICU
Inability to administer study drug within 24 hours of diagnosis of AKI according to AKI inclusion criteria
Presence of AKI in the Investigator's opinion as suggested by clinical manifestation, e.g., prolonged oliguria or severe renal dysfunction on admission without a history of CKD, for a period longer than 24 hours prior to study drug administration
Evidence of recovery from AKI based on the investigator's clinical judgement prior to randomization
Documented (biopsy proven) or suspected history of acute or sub-acute kidney diseases such as rapidly progressive glomerular nephritis (RPGN) and acute interstitial nephritis (AIN)
Participants who are thrombocytopeniat screening (platelet count <50,000 microliter) or other high risk for bleeding in the opinion of the investigator.

### Primary Objective(s):

To evaluate the dose response relationship of CD39* on renal function measured by creatinine clearance (CrCl) in participants with SA-AKI.

### Secondary Objective(s):

To evaluate the effect of CD39* versus placebo in reducing major adverse kidney events
To evaluate the effect of CD39* versus placebo in improving renal function
To evaluate the effect of CD39* versus placebo in improving overall health condition
To evaluate safety and tolerability of CD39* versus placebo

The secondary estimands are defined by the evaluation of study treatment effect on the following endpoints and summary measures:
MAKE (death, RRT, ≥25% reduction in eGFR) at Day 90: The summary measure is the odds ratio of participants with MAKE.
The weighted average of area under the time-corrected endogenous serum creatinine curve from Day 1 to Day 14 and from Day 1 to Day 30: the summary measure is the geometric mean.
The weighted average of area under the time-corrected endogenous serum cystatin-C curve from Day 1 to Day 14 and from Day 1 to Day 30: the summary measure is the geometric mean.
The weighted average of area under the time-corrected endogenous creatinine clearance curve from Day 5 to Day 14 (AUC5-14). The estimand framework is the same as primary endpoint.
Any use of RRT during the study: The summary measure is the odds ratio of participants with RRT.
RRT dependency at Day 90: The summary measure is the odds ratio of participants using RRT.
Days alive and free of RRT during the study: The summary measure is the average days alive and free of RRT.
Proportion of participants with ≥25% reduction in eGFR at Day 90: The summary measure is the odds ratio of participants with ≥25% reduction in eGFR.
KDIGO AKI stage: The summary measure is the status change from baseline in KDIGO AKI stage at Day 14.
Change of SOFA score from baseline to Day 30: The summary measure is the mean change from baseline in SOFA score.

Potential study participants will have a recent diagnosis of sepsis according to the criteria defined by The Third International Consensus Definition for Sepsis and Septic Shock (Sepsis-3) followed by a diagnosis of AKI.

Sequence listing

| SEQ | | |
|---|---|---|
| ID NO | Description | Sequence |
| 1 | human recombinant | TQNKALPENVKYGIVLDAGSSHTSLYIYKWPAEK |
| | CD39 variant | ENDTGVVHQVEECRVKGPGISKFVQKVNEIGIYLT |
| | | DCMERAREVIPRSQHQETPVYLGATAGMRLLRM |
| | | ESEELADRVLDVVERSLSNYPFDFQGARIITGQEE |
| | | GAYGWITINYLLGKFSQKNQETFGALDLGGASTQ |
| | | VTFVPQNQTIESPDNALQFRLYGKDYNVYTHSFL |
| | | CYGKDQALWQKLAKDIQVASNEILRDPCFHPGYK |
| | | KVVNVSDLYKTPCTKRFEMTLPFQQFEIQGIGNY |
| | | QQCHQSILELFNTSYCPYSQCAFNGIFLPPLQGDF |
| | | GAFSAFYFVMKFLNLTSEKVSQEKVTEMMKKFC |
| | | AQPWEEIKTSYAGVKEKYLSEYCFSGTYILSLLLQ |
| | | GYHFTADSWEHIHFIGKIQGSDAGWTLGYMLNLT |
| | | NMIPAEQPLSTPLSHST |
| 2 | human recombinant | TQNKALPENVKYGIVLDAGSSHTSLYIYKWPAEK |
| | CD39 variant | ENDTGVVHQVEECRVKGPGISKFVQKVNEIGIYLT |
| | | DCMERAMEVIPRSQHQETPVYLGATAGMRLLRM |
| | | ESEELADRVLDVVERSLSNYPFDFQGARIITGQEE |
| | | GAYGWITINYLLGKFSQKNQETFGALDLGGASTQ |
| | | VTFVPQNQTIESPDNALQFRLYGKDYNVYTHSFL |
| | | CYGKDQALWQKLAKDIQVASNEILRDPCFHPGYK |
| | | KVVNVSDLYKTPCTKRFEMTLPFQQFEIQGIGNY |
| | | QQCHQSILELFNTSYCPYSQCAFNGIFLPPLQGDF |
| | | GAFSAFYFVMKFLNLTSEKVSQEKVTEMMKKFC |
| | | AQPWEEIKTSYAGVKEKYLSEYCFSGTYILSLLLQ |
| | | GYHFTADSWEHIHFIGKIQGSDAGWTLGYMLNLT |
| | | NMIPAEQPLSTPLSHST |
| 3 | human recombinant | TQNKALPENVKYGIVLDAGSSHTSLYIYKWPAEK |
| | CD39 variant | ENDTGVVHQVEECRVKGPGISKFVQKVNEIGIYLT |
| | | DCMERAREVIPRSQHQETPVYLGATAGMRLLRM |
| | | ESEELADRVLDVVERSLSNYPFDFQGARIITGQEE |
| | | GAYGWITINYLLGKFSQKNQETFGALDLGGASTQ |
| | | VTFVPQNQTIESPDNALQFRLYGKDYNVYTHSFL |
| | | CYGKDQALWQKLAKDIQVASNEILRDPCFHPGYK |
| | | KVVNVSDLYKTPCTKRFEMTLPFQQFEIQGIGNY |
| | | QQCHQSILELFNTSYCPYSQCAFNGIFLPPLQGDF |
| | | GAFSAFYSVMKFLNLTSEKVSQEKVTEMMKKFC |
| | | AQPWEEIKTSYAGVKEKYLSEYCFSGTYILSLLLQ |
| | | GYHFTADSWEHIHFIGKIQGSDAGWTLGYMLNLT |
| | | NMIPAEQPLSTPLSHST |
| 4 | human recombinant | TQNKALPENVKYGIVLDAGSSHTSLYIYKWPAEK |
| | CD39 variant | ENDTGVVHQVEECRVKGPGISKFVQKVNEIGIYLT |
| | | DCMERAMEVIPRSQHQETPVYLGATAGMRLLRM |
| | | ESEELADRVLDVVERSLSNYPFDFQGARIITGQEE |
| | | GAYGWITINYLLGKFSQKNQETFGALDLGGASTQ |
| | | VTFVPQNQTIESPDNALQFRLYGKDYNVYTHSFL |
| | | CYGKDQALWQKLAKDIQVASNEILRDPCFHPGYK |
| | | KVVNVSDLYKTPCTKRFEMTLPFQQFEIQGIGNY |
| | | QQCHQSILELFNTSYCPYSQCAFNGIFLPPLQGDF |
| | | GAFSAFYFVMKFLNLTSEKVSQEKVTEMMKKFC |
| | | AQPWEEIKTSYAGVNEKYLSEFCFSGTYILSLLLQ |
| | | GYHFTADSWEHIHFIGKIQGSDAGWTLGYMLNLT |
| | | NMIPAEQPLSTPLSHST |
| 5 | human recombinant | TQNKALPENVKYGIVLDAGSSHTSLYIYKWPAEK |
| | CD39 variant | ENDTGVVHQVEECRVKGPGISKFVQKVNEIGIYLT |
| | | DCMERAMEVIPRSQHQETPVYLGATAGMRLLRM |
| | | ESEELADRVLDVVERSLSNYPFDFQGARIITGQEE |
| | | GAYGWITINYLLGKFSQKNQETFGALDLGGASTQ |
| | | VTFVPQNQTIESPDNALQFRLYGKDYNVYTHSFL |
| | | CYGKDQALWQKLAKDIQVASNEILRDPCFHPGYK |
| | | KVVNVSDLYKTPCTKRFEMTLPFQQFEIQGIGNY |
| | | QQCHQSILELFNTSYCPYSQCAFNGIFLPPLQGDF |
| | | GAFSAFYFVMKFLNLTSEKVSQEKVTEMMKKFC |
| | | AQPWEEIKTSYAGVKEKYLSEFCFSGTYILSLLLQ |
| | | GYHFTADSWEHIHFIGKIQGSDAGWTLGYMLNLT |
| | | NMIPAEQPLSTPLSHST |
| 6 | human recombinant | TQNKALPENVKYGIVLDAGSSHTSLYIYKWPAEK |
| | CD39 variant | ENDTGVVHQVEECRVKGPGISKFVQKVNEIGIYLT |
| | | DCMERAREVIPRSQHQETPVYLGATAGMRLLRM |
| | | ESEELADRVLDVVERSLSNYPFDFQGARIITGQEE |
| | | GAYGWITINYLLGKFSQKNQETFGALDLGGASTQ |
| | | VTFVPQNQTIESPDNALQFRLYGKDYNVYTHSFL |
| | | CYGKDQALWQKLAKDIQVASNEILRDPCFHPGYK |
| | | KVVNVSDLYKTPCTKRFEMTLPFQQFEIQGIGNY |
| | | QQCHQSILELFNTSYCPYSQCAFNGIFLPPLQGDF |
| | | GAFSAFYFVMKFLNLTSEKVSQEKVTEMMKKFC |
| | | AQPWEEIKTSYAGVKEKYLSEFCFSGTYILSLLQQ |
| | | GYHFTADSWEHIHFIGKIQGSDAGWTLGYMLNLT |
| | | NMIPAEQPLSTPLSHST |
| 7 | human recombinant | TQNKALPENVKYGIVLDAGSSHTSLYIYKWPAEK |
| | CD39 variant | ENDTGVVHQVEECRVKGPGISKFVQKVNEIGIYLT |
| | | DCMERAMEVIPRSQHQETPVYLGATAGMRLLRM |
| | | ESEELADRVLDVVERSLSNYPFDFQGARIITGQEE |
| | | GAYGWITINYLLGKFSQKNQETFGALDLGGASTQ |
| | | VTFVPQNQTIESPDNALQFRLYGKDYNVYTHSFL |
| | | CYGKDQALWQKLAKDIQVASNEILRDPCFHPGYK |
| | | KVVNVSDLYKTPCTKRFEMTLPFQQFEIQGIGNY |
| | | QQCHQSILELFNTSYCPYSQCAFNGIFLPPLQGDF |
| | | GAFSAFYFVMKFLNLTSEKVSQEKVTEMMKKFC |
| | | AQPWEEIKTSYAGVKEKYLSEFCFSGTYILSLLQQ |
| | | GYHFTADSWEHIHFIGKIQGSDAGWTLGYMLNLT |
| | | NMIPAEQPLSTPLSHST |
| 8 | human recombinant | TQNKALPENVKYGIVLDAGSSHTSLYIYKWPAEK |
| | CD39 variant | ENDTGVVHQVEECRVKGPGISKFVQKVNEIGIYLT |
| | | DCMERAMEVIPRSQHQETPVYLGATAGMRLLRM |
| | | ESEELADRVLDVVERSLSNYPFDFQGARIITGQEE |
| | | GAYGWITINYLLGKFSQKNQETFGALDLGGASTQ |
| | | VTFVPQNQTIESPDNALQFRLYGKDYNVYTHSFL |
| | | CYGKDQALWQKLAKDIQVASNEILRDPCFHPGYK |
| | | KVVNVSDLYKTPCTKRFEMTLPFQQFEIQGIGNY |
| | | QQCHQSILELFNTSYCPYSQCAFNGIFLPPLQGDF |
| | | GAFSAFYSVMKFLNLTSEKVSQEKVTEMMKKFC |
| | | AQPWEEIKTSYAGVKEKYLSEYCFSGTYILSLLLQ |
| | | GYHFTADSWEHIHFIGKIQGSDAGWTLGYMLNLT |
| | | NMIPAEQPLSTPLSHST |
| 9 | human recombinant | APTSSSTQNKALPENVKYGIVLDAGSSHTSLYIYK |
| | CD39 variant | WPAEKENDTGVVHQVEECRVKGPGISKFVQKVN |
| | | EIGIYLTDCMERAMEVIPRSQHQETPVYLGATAG |
| | | MRLLRMESEELADRVLDVVERSLSNYPFDFQGAR |
| | | IITGQEEGAYGWITINYLLGKFSQKNQETFGALDL |
| | | GGASTQVTFVPQNQTIESPDNALQFRLYGKDYNV |
| | | YTHSFLCYGKDQALWQKLAKDIQVASNEILRDPC |
| | | FHPGYKKVVNVSDLYKTPCTKRFEMTLPFQQFEI |
| | | QGIGNYQQCHQSILELFNTSYCPYSQCAFNGIFLPP |
| | | LQGDFGAFSAFYFVMKFLNLTSEKVSQEKVTEM |
| | | MKKFCAQPWEEIKTSYAGVKEKYLSEFCFSGTYIL |
| | | SLLLQGYHFTADSWEHIHFIGKIQGSDAGWTLGY |
| | | MLNLTNMIPAEQPLSTPLSHST |
| 10 | human recombinant | APTSSSTQNKALPENVKYGIVLDAGSSHTSLYIYK |
| | CD39 variant | WPAEKENDTGVVHQVEECRVKGPGISKFVQKVN |
| | | EIGIYLTDCMERAREVIPRSQHQETPVYLGATAGM |
| | | RLLRMESEELADRVLDVVERSLSNYPFDFQGARII |
| | | TGQEEGAYGWITINYLLGKFSQKNQETFGALDLG |
| | | GASTQVTFVPQNQTIESPDNALQFRLYGKDYNVY |
| | | THSFLCYGKDQALWQKLAKDIQVASNEILRDPCF |
| | | HPGYKKVVNVSDLYKTPCTKRFEMTLPFQQFEIQ |
| | | GIGNYQQCHQSILELFNTSYCPYSQCAFNGIFLPPL |
| | | QGDFGAFSAFYFVMKFLNLTSEKVSQEKVTEMM |
| | | KKFCAQPWEEIKTSYAGVKEKYLSEFCFSGTYILS |
| | | LLQQGYHFTADSWEHIHFIGKIQGSDAGWTLGYM |
| | | LNLTNMIPAEQPLSTPLSHST |
| 11 | human recombinant | APTSSSTQNKALPENVKYGIVLDAGSSHTSLYIYK |
| | CD39 variant | WPAEKENDTGVVHQVEECRVKGPGISKFVQKVN |
| | | EIGIYLTDCMERAMEVIPRSQHQETPVYLGATAG |
| | | MRLLRMESEELADRVLDVVERSLSNYPFDFQGAR |
| | | IITGQEEGAYGWITINYLLGKFSQKNQETFGALDL |
| | | GGASTQVTFVPQNQTIESPDNALQFRLYGKDYNV |
| | | YTHSFLCYGKDQALWQKLAKDIQVASNEILRDPC |
| | | FHPGYKKVVNVSDLYKTPCTKRFEMTLPFQQFEI |
| | | QGIGNYQQCHQSILELFNTSYCPYSQCAFNGIFLPP |
| | | LQGDFGAFSAFYFVMKFLNLTSEKVSQEKVTEM |
| | | MKKFCAQPWEEIKTSYAGVKEKYLSEFCFSGTYIL |
| | | SLLQQGYHFTADSWEHIHFIGKIQGSDAGWTLGY |
| | | MLNLTNMIPAEQPLSTPLSHST |
| 12 | human recombinant | APTTQNKALPENVKYGIVLDAGSSHTSLYIYKWP |
| | CD39 variant | AEKENDTGVVHQVEECRVKGPGISKFVQKVNEIG |
| | | IYLTDCMERAREVIPRSQHQETPVYLGATAGMRL |
| | | LRMESEELADRVLDVVERSLSNYPFDFQGARIITG |
| | | QEEGAYGWITINYLLGKFSQKNQETFGALDLGGA |
| | | STQVTFVPQNQTIESPDNALQFRLYGKDYNVYTH |
| | | SFLCYGKDQALWQKLAKDIQVASNEILRDPCFHP |
| | | GYKKVVNVSDLYKTPCTKRFEMTLPFQQFEIQGI |
| | | GNYQQCHQSILELFNTSYCPYSQCAFNGIFLPPLQ |
| | | GDFGAFSAFYFVMKFLNLTSEKVSQEKVTEMMK |
| | | KFCAQPWEEIKTSYAGVKEKYLSEFCFSGTYILSL |
| | | LQQGYHFTADSWEHIHFIGKIQGSDAGWTLGYML |
| | | NLTNMIPAEQPLSTPLSHST |
| 13 | human recombinant | APTTQNKALPENVKYGIVLDAGSSHTSLYIYKWP |
| | CD39 variant | AEKENDTGVVHQVEECRVKGPGISKFVQKVNEIG |
| | | IYLTDCMERAMEVIPRSQHQETPVYLGATAGMRL |
| | | LRMESEELADRVLDVVERSLSNYPFDFQGARIITG |
| | | QEEGAYGWITINYLLGKFSQKNQETFGALDLGGA |
| | | STQVTFVPQNQTIESPDNALQFRLYGKDYNVYTH |
| | | SFLCYGKDQALWQKLAKDIQVASNEILRDPCFHP |
| | | GYKKVVNVSDLYKTPCTKRFEMTLPFQQFEIQGI |
| | | GNYQQCHQSILELFNTSYCPYSQCAFNGIFLPPLQ |
| | | GDFGAFSAFYFVMKFLNLTSEKVSQEKVTEMMK |
| | | KFCAQPWEEIKTSYAGVKEKYLSEFCFSGTYILSL |
| | | LQQGYHFTADSWEHIHFIGKIQGSDAGWTLGYML |
| | | NLTNMIPAEQPLSTPLSHST |
| 14 | human recombinant | APTTQNKALPENVKYGIVLDAGSSHTSLYIYKWP |
| | CD39 variant | AEKENDTGVVHQVEECRVKGPGISKFVQKVNEIG |
| | | IYLTDCMERAMEVIPRSQHQETPVYLGATAGMRL |
| | | LRMESEELADRVLDVVERSLSNYPFDFQGARIITG |
| | | QEEGAYGWITINYLLGKFSQKNQETFGALDLGGA |
| | | STQVTFVPQNQTIESPDNALQFRLYGKDYNVYTH |
| | | SFLCYGKDQALWQKLAKDIQVASNEILRDPCFHP |
| | | GYKKVVNVSDLYKTPCTKRFEMTLPFQQFEIQGI |
| | | GNYQQCHQSILELFNTSYCPYSQCAFNGIFLPPLQ |
| | | GDFGAFSAFYFVMKFLNLTSEKVSQEKVTEMMK |
| | | KFCAQPWEEIKTSYAGVKEKYLSEYCFSGTYILSL |
| | | LLQGYHFTADSWEHIHFIGKIQGSDAGWTLGYML |
| | | NLTNMIPAEQPLSTPLSHST |
| 15 | human recombinant | APTTQNKALPENVKYGIVLDAGSSHTSLYIYKWP |
| | CD39 variant | AEKENDTGVVHQVEECRVKGPGISKFVQKVNEIG |
| | | IYLTDCMERAMEVIPRSQHQETPVYLGATAGMRL |
| | | LRMESEELADRVLDVVERSLSNYPFDFQGARIITG |
| | | QEEGAYGWITINYLLGKFSQKNQETFGALDLGGA |
| | | STQVTFVPQNQTIESPDNALQFRLYGKDYNVYTH |
| | | SFLCYGKDQALWQKLAKDIQVASNEILRDPCFHP |
| | | GYKKVVNVSDLYKTPCTKRFEMTLPFQQFEIQGI |
| | | GNYQQCHQSILELFNTSYCPYSQCAFNGIFLPPLQ |
| | | GDFGAFSAFYSVMKFLNLTSEKVSQEKVTEMMK |
| | | KFCAQPWEEIKTSYAGVKEKYLSEYCFSGTYILSL |
| | | LLQGYHFTADSWEHIHFIGKIQGSDAGWTLGYML |
| | | NLTNMIPAEQPLSTPLSHST |
| 16 | human recombinant | APTSSSTKKTQLTSSGTQNKALPENVKYGIVLDAG |
| | CD39 variant | SSHTSLYIYKWPAEKENDTGVVHQVEECRVKGPG |
| | | ISKFVQKVNEIGIYLTDCMERAREVIPRSQHQETP |
| | | VYLGATAGMRLLRMESEELADRVLDVVERSLSN |
| | | YPFDFQGARIITGQEEGAYGWITINYLLGKFSQKN |
| | | QETFGALDLGGASTQVTFVPQNQTIESPDNALQFR |
| | | LYGKDYNVYTHSFLCYGKDQALWQKLAKDIQVA |
| | | SNEILRDPCFHPGYKKVVNVSDLYKTPCTKRFEM |
| | | TLPFQQFEIQGIGNYQQCHQSILELFNTSYCPYSQC |
| | | AFNGIFLPPLQGDFGAFSAFYFVMKFLNLTSEKVS |
| | | QEKVTEMMKKFCAQPWEEIKTSYAGVKEKYLSE |
| | | YCFSGTYILSLLLQGYHFTADSWEHIHFIGKIQGSD |
| | | AGWTLGYMLNLTNMIPAEQPLSTPLSHST |
| 17 | human recombinant | APTSSSTKKTQLTSSTQNKALPENVKYGIVLDAGS |
| | CD39 variant | SHTSLYIYKWPAEKENDTGVVHQVEECRVKGPGI |
| | | SKFVQKVNEIGIYLTDCMERAMEVIPRSQHQETPV |
| | | YLGATAGMRLLRMESEELADRVLDVVERSLSNY |
| | | PFDFQGARIITGQEEGAYGWITINYLLGKFSQKNQ |
| | | ETFGALDLGGASTQVTFVPQNQTIESPDNALQFRL |
| | | YGKDYNVYTHSFLCYGKDQALWQKLAKDIQVAS |
| | | NEILRDPCFHPGYKKVVNVSDLYKTPCTKRFEMT |
| | | LPFQQFEIQGIGNYQQCHQSILELFNTSYCPYSQCA |
| | | FNGIFLPPLQGDFGAFSAFYFVMKFLNLTSEKVSQ |
| | | EKVTEMMKKFCAQPWEEIKTSYAGVNEKYLSEF |
| | | CFSGTYILSLLLQGYHFTADSWEHIHFIGKIQGSDA |
| | | GWTLGYMLNLTNMIPAEQPLSTPLSHST |
| 18 | human recombinant | APTSSSTQNKALPENVKYGIVLDAGSSHTSLYIYK |
| | CD39 variant | WPAEKENDTGVVHQVEECRVKGPGISKFVQKVN |
| | | EIGIYLTDCMERAMEVIPRSQHQETPVYLGATAG |
| | | MRLLRMESEELADRVLDVVERSLSNYPFDFQGAR |
| | | IITGQEEGAYGWITINYLLGKFSQKNQETFGALDL |
| | | GGASTQVTFVPQNQTIESPDNALQFRLYGKDYNV |
| | | YTHSFLCYGKDQALWQKLAKDIQVASNEILRDPC |
| | | FHPGYKKVVNVSDLYKTPCTKRFEMTLPFQQFEI |
| | | QGIGNYQQCHQSILELFNTSYCPYSQCAFNGIFLPP |
| | | LQGDFGAFSAFYSVMKFLNLTSEKVSQEKVTEM |
| | | MKKFCAQPWEEIKTSYAGVKEKYLSEYCFSGTYI |
| | | LSLLLQGYHFTADSWEHIHFIGKIQGSDAGWTLG |
| | | YMLNLTNMIPAEQPLSTPLSHST |
| 19 | human recombinant | APTTQNKALPENVKYGIVLDAGSSHTSLYIYKWP |
| | CD39 variant | AEKENDTGVVHQVEECRVKGPGISKFVQKVNEIG |
| | | IYLTDCMERAMEVIPRSQHQETPVYLGATAGMRL |
| | | LRMESEELADRVLDVVERSLSNYPFDFQGARIITG |
| | | QEEGAYGWITINYLLGKFSQKNQETFGALDLGGA |
| | | STQVTFVPQNQTIESPDNALQFRLYGKDYNVYTH |
| | | SFLCYGKDQALWQKLAKDIQVASNEILRDPCFHP |
| | | GYKKVVNVSDLYKTPCTKRFEMTLPFQQFEIQGI |
| | | GNYQQCHQSILELFNTSYCPYSQCAFNGIFLPPLQ |
| | | GDFGAFSAFYFVMKFLNLTSEKVSQEKVTEMMK |
| | | KFCAQPWEEIKTSYAGVKEKYLSEFCFSGTYILSL |
| | | LLQGYHFTADSWEHIHFIGKIQGSDAGWTLGYML |
| | | NLTNMIPAEQPLSTPLSHST |
| 20 | human recombinant | APTTQNKALPENVKYGIVLDAGSSHTSLYIYKWP |
| | CD39 variant | AEKENDTGVVHQVEECRVKGPGISKFVQKVNEIG |
| | | IYLTDCMERAREVIPRSQHQETPVYLGATAGMRL |
| | | LRMESEELADRVLDVVERSLSNYPFDFQGARIITG |
| | | QEEGAYGWITINYLLGKFSQKNQETFGALDLGGA |
| | | STQVTFVPQNQTIESPDNALQFRLYGKDYNVYTH |
| | | SFLCYGKDQALWQKLAKDIQVASNEILRDPCFHP |
| | | GYKKVVNVSDLYKTPCTKRFEMTLPFQQFEIQGI |
| | | GNYQQCHQSILELFNTSYCPYSQCAFNGIFLPPLQ |
| | | GDFGAFSAFYFVMKFLNLTSEKVSQEKVTEMMK |
| | | KFCAQPWEEIKTSYAGVKEKYLSEYCFSGTYILSL |
| | | LLQGYHFTADSWEHIHFIGKIQGSDAGWTLGYML |
| | | NLTNMIPAEQPLSTPLSHST |
| 21 | human recombinant | APTTQNKALPENVKYGIVLDAGSSHTSLYIYKWP |
| | CD39 variant | AEKENDTGVVHQVEECRVKGPGISKFVQKVNEIG |
| | | IYLTDCMERAREVIPRSQHQETPVYLGATAGMRL |
| | | LRMESEELADRVLDVVERSLSNYPFDFQGARIITG |
| | | QEEGAYGWITINYLLGKFSQKNQETFGALDLGGA |
| | | STQVTFVPQNQTIESPDNALQFRLYGKDYNVYTH |
| | | SFLCYGKDQALWQKLAKDIQVASNEILRDPCFHP |
| | | GYKKVVNVSDLYKTPCTKRFEMTLPFQQFEIQGI |
| | | GNYQQCHQSILELFNTSYCPYSQCAFNGIFLPPLQ |
| | | GDFGAFSAFYSVMKFLNLTSEKVSQEKVTEMMK |
| | | KFCAQPWEEIKTSYAGVKEKYLSEYCFSGTYILSL |
| | | LLQGYHFTADSWEHIHFIGKIQGSDAGWTLGYML |
| | | NLTNMIPAEQPLSTPLSHST |
| | | MEDTKESNVKTFCSKNILAILGFSSIIAVIALLAVG |
| 22 | human recombinant | LTQNKALPENVKYGIVLDAGSSHTSLYIYKWPAE |
| | CD39 | KENDTGVVHQVEECRVKGPGISKFVQKVNEIGIY |
| | | LTDCMERAREVIPRSQHQETPVYLGATAGMRLLR |
| | | MESEELADRVLDVVERSLSNYPFDFQGARIITGQE |
| | | EGAYGWITINYLLGKFSQKTRWFSIVPYETNNQET |
| | | FGALDLGGASTQVTFVPQNQTIESPDNALQFRLY |
| | | GKDYNVYTHSFLCYGKDQALWQKLAKDIQVASN |
| | | EILRDPCFHPGYKKVVNVSDLYKTPCTKRFEMTL |
| | | PFQQFEIQGIGNYQQCHQSILELFNTSYCPYSQCAF |
| | | NGIFLPPLQGDFGAFSAFYFVMKFLNLTSEKVSQE |
| | | KVTEMMKKFCAQPWEEIKTSYAGVKEKYLSEYC |
| | | FSGTYILSLLLQGYHFTADSWEHIHFIGKIQGSDAG |
| | | WTLGYMLNLTNMIPAEQPLSTPLSHSTYVFLMVL |
| | | FSLVLFTVAIIGLLIFHKPSYFWKDMV |

1. A method of treating or preventing AKI in a subject in need thereof comprising administering a recombinant CD39.

2. The method according to claim 1, wherein the AKI is severe AKI.

3. The method according to claim 1, wherein at least one symptom of AKI in a subject is reduced or abolished.

4. The method according to any one of the above claims, wherein the severity of AKI is reduced by at least one stage, as defined by modified AKI Network (AKIN) criteria for serum creatinine.

5. The method according to any one of the above claims, wherein the recombinant CD39 is to be administered at dose of from about 0.1 mg/kg to about 10 mg/kg of the recombinant CD39.

6. The method according to claim 5, wherein the recombinant CD39 administered at a dose of about 4 mg/kg of the recombinant CD39.

7. The method according to claim 5, wherein the recombinant CD39 is administered at a dose of about 5 mg/kg of the recombinant CD39.

8. The method according to any one of the above claims, wherein the recombinant CD39 is administered intravenously.

9. The method according to any one of the above claims, wherein the recombinant CD39 is administered within a number of hours after AKI is first diagnosed for said subject.

10. The method according to any one of the above claims, wherein the recombinant CD39 is administered within about 48 hours, or less, after AKI is first diagnosed for said subject (e.g., within 12 hours, within 24 hours, or within 36 hours).

11. The method according to any one of the above claims, wherein the AKI is sepsis associated acute kidney injury (SA-AKI).

12. The method according to any of the above claims, wherein the AKI is cardiac surgery associated AKI (CSA-AKI).

13. The method according to any of the above claims, wherein the method comprises preventing or treating one or more symptoms associated with AKI.

14. The method according to any one of the above claims, wherein the recombinant CD39 has an amino acid sequence selected from the group consisting of SEQ ID NOs: 1 to 21.

15. A method of administering a recombinant CD39 having an amino acid sequence selected from the group consisting of SEQ ID NOs: 1 to 21 at dose of from about 0.1 mg/kg to about 10 mg/kg of the recombinant CD39.

16. (canceled)

17. The method according claim 15, wherein the recombinant CD39 has an amino acid sequence of SEQ ID NO: 21.

18. The method according to any one of the above claims, wherein the recombinant CD39 is administered as monotherapy to said subject.

19. The method according to any one of the above claims, wherein the recombinant CD39 is co-administered with an additional therapeutic agent.

20. The method according to claim 19, wherein the recombinant CD39 is co-administered with an additional therapeutic agent selected from a steroid, a metalloproteinase inhibitor, a serine protease inhibitor, a topical anesthetic emulsion, a spreading factor inhibitor, an anti-nausea agent, or an antibiotic.
